(19) United States
(12) Patent Application Publication
(10) Pub. No.: US 2022/0160493 A1
(43) Pub. Date: May 26, 2022

(54) Intraocular Refractive Phakic Lens and Method

(71) Applicant: Vladimir Feingold, LAKE FOREST, CA (US)

(72) Inventor: Vladimir Feingold, LAKE FOREST, CA (US)

(21) Appl. No.: 17/584,014

(22) Filed: Jan. 25, 2022

Related U.S. Application Data
(63) Continuation of application No. PCT/US2020/037306, filed on Jun. 11, 2020.
(60) Provisional application No. 62/904,640, filed on Sep. 23, 2019.

(51) Int. Cl. A61F 2/16

(52) U.S. Cl. CPC A61F 2/1601; A61F 2/161; A61F 2002/1682; A61F 2002/1696; A61F 2002/16903; A61F 2002/16905; A61F 2002/1683

(57) ABSTRACT

Described is a refractive phakic lens that has haptics extending away from the optical portion and in which the haptics have a compressible and release portion in the form of reversed undulation shape and at the end of the reversed undulation is a haptic pad that contacts the sulcus. Also, there is a rim structure on the posterior surface of the optic member which extend circumferentially but has interruption. Also, there is an opening at the optical axis of the lens member. Thereby fluid can flow through the opening and through the interruptions.

### BACKGROUND OF THE INVENTION

An intraocular lens (IOL) is a lens implanted in the eye to treat large refractive errors. The IOLs usually consist of small optics with side structures to hold the lens in place. There are two types of IOLs. One type is inserted into the capsular bag replacing the natural crystalline lens. The other type, known as a phakic IOL is placed within the eye under the iris without removing the existing natural lens.

### BRIEF DESCRIPTION OF THE FIGURES

FIG. 1 is an anterior perspective view of an embodiment of the invention having an exemplary reduced vertical dimension to an oval shape and having an optic portion comprising a lens member and a darkened concentric surround member and having a haptic carrier portion from which the haptic assemblies extend, the haptic assemblies having a flexible resilient portion of reversed undulations and a pad portion, the reversed undulations being in the same rotational orientation or order, and a fluid flow opening is centered through the lens and rims having interruptions to provide flow paths.

FIG. 2 is a posterior perspective view of the embodiment of FIG. 1 showing a rim structure with interruption for fluid flow in conjunction with the opening in the center of the lens.

FIG. 3 is a top and bottom side/edge view also referred to as a horizontal side/edge view of the embodiment of FIGS. 1 and 2.

FIG. 4. is a left and right side/edge view also referred to as a vertical side/edge view of the embodiment of FIGS. 1 and 2.

FIG. 5. is an anterior view (facing outwardly of the eye) also considered as a plan view of the embodiment of FIGS. 1 and 2.

FIG. 6 is a posterior view (facing inwardly of the eye) considered an inverted view of FIG. 5, of the embodiment of FIGS. 1 and 2.

FIG. 7 is sectional view along line A-A′ of FIG. 5.

FIG. 8 is a sectional view along line B-B′ of FIG. 5.

FIG. 9 is a sectional view along line E-E′ of FIG. 5

FIG. 10 is an anterior perspective view of an alternative embodiment of the invention referred to for convenience as a full circle configuration having an optic portion in full circle comprising a lens member and a darkened member extending concentrically around the lens member and having a set of haptic assemblies the haptic assemblies having flexible resilient portions of undulating form defining a reversed undulation and terminal pad portions, the reversed undulations being in paired mirror image lateral extension, and an opening is centered through the lens member and having interrupted rims defining flow spaces such that the flow spaces and the opening allow fluid flow.

FIG. 11 is a posterior perspective view of the embodiment of FIG. 1 showing a rim structure with interruption for fluid flow in conjunction with the opening in the center of the lens.

FIG. 12 is a top and bottom side/edge view also referred to as a horizontal side/edge view of the embodiment of FIGS. 10 and 11.

FIG. 13. is a left and right side/edge view also referred to as a vertical side/edge view of the embodiment of FIGS. 10 and 11.

FIG. 14. is an anterior view (facing outwardly of the eye) also considered as a plan view of the embodiment of FIGS. 10 and 11.

FIG. 15 is a sectional view along line A-A′ of FIG. 14.

FIG. 16 is a sectional view along line B-B′ of FIG. 14.

FIG. 17 is a sectional view along line E-E′ of FIG. 14

FIG. 18 is a schematic view of a human eye showing the subject refractive phakic lens of this description in a typical installed position.

FIGS. 19-22 show an embodiment of the subject refractive phakic lens in which the haptics extend angularly posteriorly from the optic portion.

FIG. 23 is an exemplary movement of the haptic from its unstressed position in contact with the sulcus to a stressed, compressed position.

FIG. 24 is a schematic illustration to see the general location of the subject lens in the eye looking in from the front in which the IOL is overlaid on the eye for convenient viewing.

### DETAILED DESCRIPTION

In the following description variations of exemplary embodiments will be described and illustrated; it is not intended to be an exhaustive description.

For purposes of clarity some descriptive and geometric designations are used. For the intraocular lens and its subparts as described herein, there is a front facing surface also referred to as an anterior (meaning facing outwardly of the eye) surface and an inner facing surface referred to as a posterior (meaning facing inwardly of the eye) surface. In general the term anterior means a direction toward the front or in front of some other part and the term posterior means a direction toward the back or behind some other part. The term “top” refers to the view looking down from above a lens as installed in the eye. The term “bottom” refers to the view looking up from below a lens as installed in the eye. The term “side” refers to the view looking horizontally from a side of the lens as installed in the eye. The term “horizontal” refers to the horizontal direction across the eye from side-to-side. The term “vertical” refers to the vertical direction of an eye from top to bottom. The term “edge” means looking in from the side, top or bottom, as the case may be.

The present invention is in the technology of the intraocular lens (IOL) in the particular aspect of that technology known as the posterior chamber phakic intraocular lens (IOL). In this technology, there are a plentitude of variables both for the function of the implant and for the means and method for its implantation. The present description of exemplary embodiments for the IOL configuration and for its method of implantation include but are not limited to the following:

1) A configuration for allowing flow of fluids between the posterior and anterior chambers of the eye, while avoiding increased risk of impeding visual acuity and the risk of increasing intraocular pressure:
a) by including a rim member around the s periphery of an optic portion of the IOL which seats on the periphery of the crystalline (natural) lens or zonulas thereby reducing the risk of cataract in the crystalline lens that may impede visual acuity;
b) providing a rim having lateral interruptions, (openings oriented to the vertical and horizontal lens axis) that improve fluid exchange;
c) providing an opening at the center of an optic element of the optics portion, preferably which is elongated along the astigmatic axis and/or the steepest meridian whereby flow of fluid is allowed around the optic portion between the posterior and anterior chambers of the eye by passing through the opening at the optic element center and through the interruptions in the rim.

2) A haptic assembly configuration and positioning of a plurality of the haptic assemblies to accomplish improved maintenance of the optic portion position aligned with the optical axis of the subject eye:
a) the haptic assembly having an application in which correct orientation is readily apparent in the implantation procedure by a visual indicator;
b) the haptic assembly having an orientation and structure to avoid or limit rotational force vectors that may be applied to the haptics and thereby cause the IOL to rotate;
c) the haptic assembly having a flexible resilient portion (also referred to as elastically deformable) that functions to be compressed and expanded in a nominally radial direction such that rotational force vectors are omitted or minimized and having a sulcus contact pad (also called a haptic pad or haptic terminal pad) that has a minimized or reduced rotational and/or lateral movement response from pressure asserted by or released by its contact with the sulcus, which flexible resilient portion is formed as consecutive oppositely facing undulations referred to as reversed undulations which extend laterally oppositely whereby opposing rotational force vectors are subtracted;

3) An embodiment of providing the haptic assemblies to extend radially angularly downwardly from the optic portion (exemplary range of about 5 degrees to 10 degrees) such that increased pressure by the sulcus contact will cause the haptic assemblies to lift the optic portion way from the crystalline lens to allow an accommodation of vision.

4) An embodiment that has an ovulated shape of the optic portion by reducing its vertical dimension which results in a smaller vertical height, less material to a lower total volume and bulk of material and a lesser vertical height such that it occupies less space when folded for insertion thereby allowing a smaller incision for the implantation process, but without any optical consequences.

5) The optic portion having a dark zone or light blocking surface as a ring around the outer diameter of the optic element of the IOL to reduce the optic element such as to block light rays on the periphery of the optic portion reducing glare and halos around lights as seen.

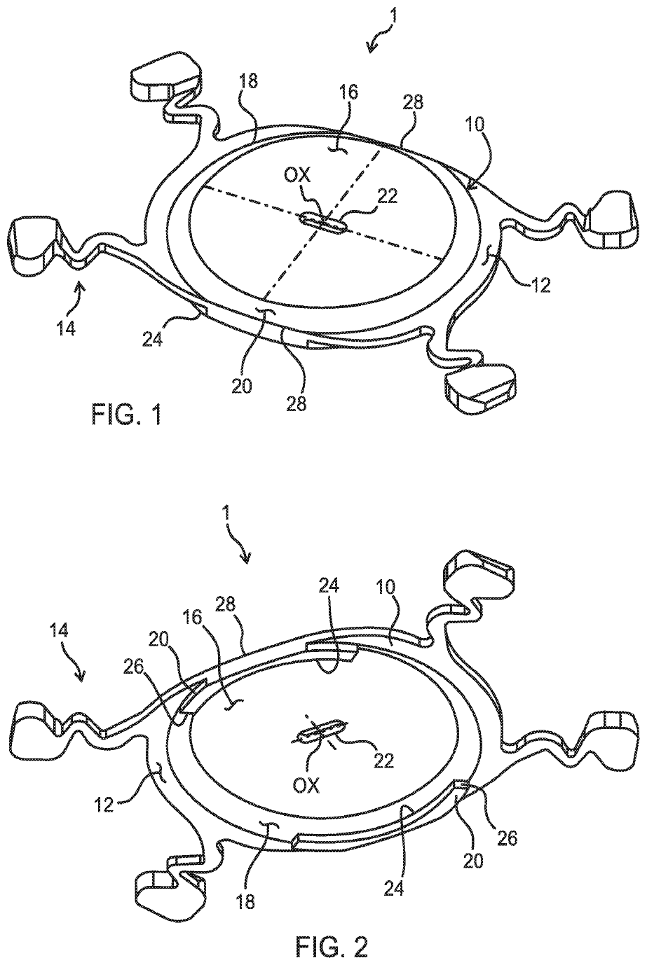

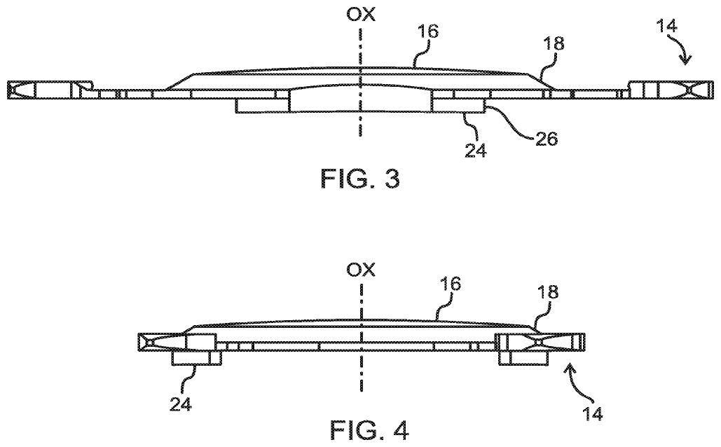

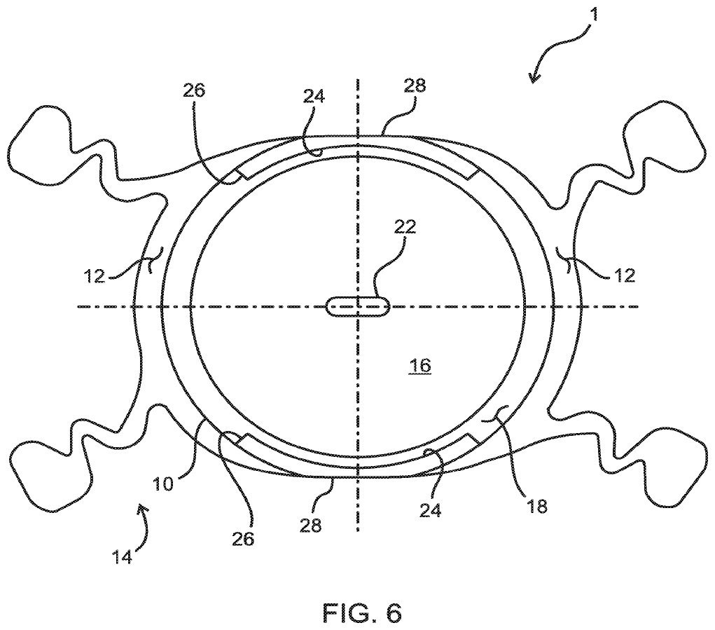

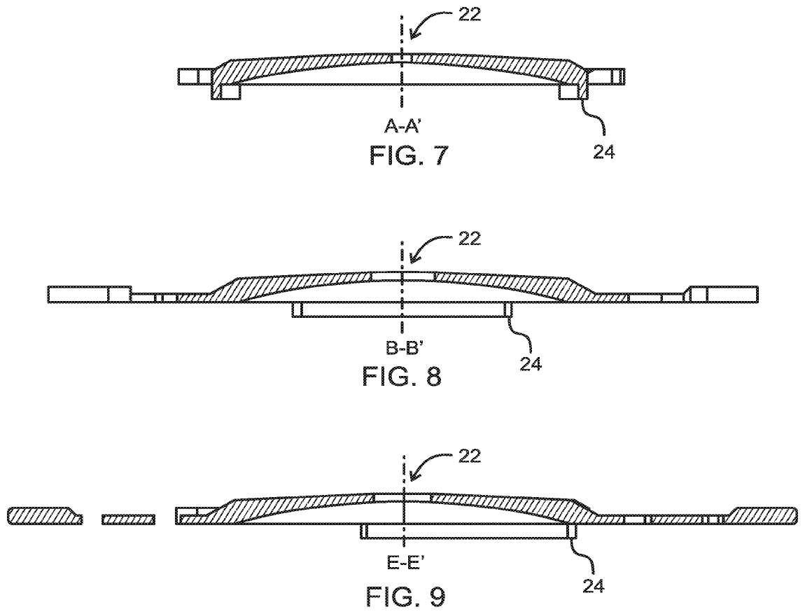

FIGS. 1-9 illustrate an embodiment of a posterior chamber phakic intraocular lens (IOL) 1 having an optic portion 10 and haptic bases 12 which are oppositely positioned at horizontal sides or edges, and haptic assemblies 14 extending in pairs from the haptic bases 12. For terminology of the structure, it is seen that the IOL 1 has a posterior side, which faces inwardly of the eye and an anterior side that faces outwardly of the eye. When viewed to its natural positioning in the eye the IOL 1 has a vertical height and a horizontal length, and it has sides being to the left and right defining vertical edges and it has sides extending along the top and bottom defining horizontal edges. The optic portion 10 is comprised of an optic element 16 and a surround ring 18 which is darkened on its upper surface or alternatively through its thickness or optically diverting or optically blocking light and the upper surface being sloped away from the optic element 16 (as seen in FIGS. 3, 4, 7, 8 and 9) which blocks the incoming light. Centrally of the optic element 16 is an opening 22, being of exemplary slot shape symmetrically around the optical axis OX, preferably extending horizontally. Rim members 24 extend peripherally around the posterior surface of the optic portion 10 with interruptions 26 in the extent of the rim members 24. The interruptions 26 in the rim members 24 define flow openings for fluid flow in conjunction with the opening slot 22. The rim members 24 have the function to hold the IOL above the crystalline lens so as to allow the flow openings to be present.

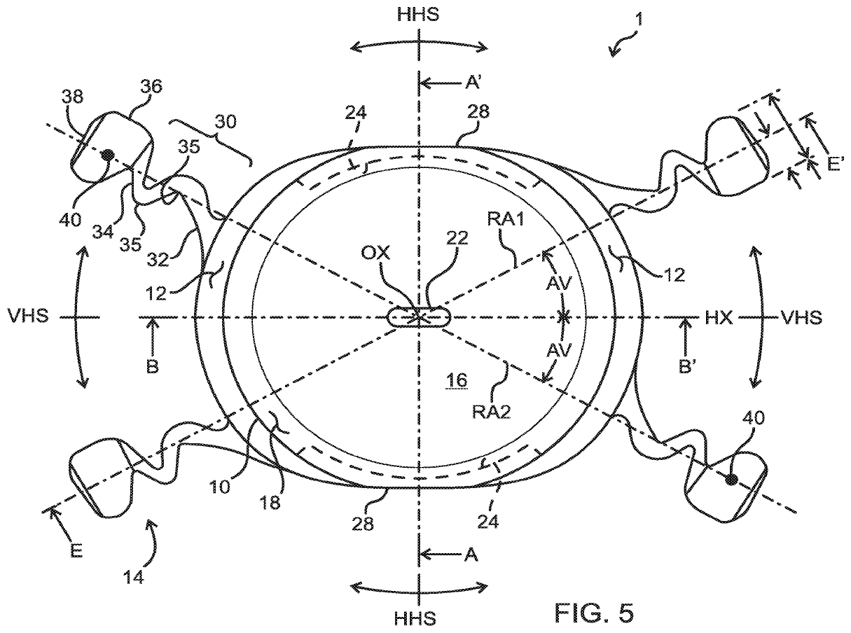

The optical axis OX defines the optical center of the optic element16, and as will be appreciated defines an axial center for the entire IOL 1. In the present embodiment, the surround ring 18 has a flattened portion 28 along its horizontal curvatures (at its top and bottom as seen in the figures) to flatten the circularity of the structure thereby reducing its height dimension and overall volume. The haptic bases 12 are attached to the optic portion 10 and extend symmetrically oppositely on each of the outer sides (left and right sides as seen in the figures) also referred to as the vertical sides or vertically extending sides. The haptic bases 12 in their upper and lower extension around the surround ring 18, thin to merge into the flattened portions 28 of the surround ring 18. The haptic assemblies 14 extend from the optic portion 10 (more specifically in this embodiment, from the haptic bases 12). There are four haptic assemblies 14. They each extend nominally along a radial line or radial axis RA (being a pair, RA1 and RA2 more detailed below). Each haptic assembly 14 comprises a spring element also referred to as a reversed undulation 30 which is constructed to functionally operate along its RA in resilient flexibility to compress or expand from an unflexed original posture. The reversed undulation 30 in the present exemplary structure comprises a pair of laterally oppositely extending portions each being an undulation and together defined as a reversed undulation. A first or lower undulation 32 extending to one side of the axis RA and a second or higher undulation 34 extending to the opposite side of the axis RA, the undulations 32 and 34 reconsidered to be nominally symmetrically opposite relative to the axis RA, although that symmetry need not be exact. In this embodiment it can be seen that the undulations 32 and 34 have sharp direction change in the nature of a bellows shape. The term bellows in this embodiment can be seen as having a distinct change of direction almost a fold point such as in a bellows flex, wherein at a point of direction change 35 like an elbow rotation is readily available to accept the compression and expansion. The reversed undulation 30 commences from the haptic base 12 extending away from the optical axis OX along the axis RA and then connects to a haptic pad 36. The haptic pad 36 has an end surface 38 also called a haptic pad sulcus surface, that is that surface 38 is where the pressure contact from the sulcus is imposed onto and released from the haptics The positioning of the haptic assemblies 14 is set to provide symmetrical interaction upon the IOL in use by the contact with the sulcus, and therefore they are positioned symmetrical around the IOL. That symmetry is defined by the radial axis RA as being a pair of the radial axes RA1 and RA2 which cross at the optical axis OX. The axes RA1 and RA2 each extend at a selected equal angular relationship AV to a horizontal axis HX and an angle of the right angle difference to vertical axis VX which run through the optical axis OX. That angular relationship may be selected according to any desired haptic posturing and interfunction of the haptic with contact to the sulcus as described in more detail below. Each of the haptic assemblies 14 therefore extend along the axes RA1 And RA2 in axial pairs outwardly such that they are arranged symmetrically as defining paired vertical haptic symmetry VHS and paired horizontal haptic symmetry HHS as indicated schematically in FIG. 5.

The haptic assemblies have a thickness that will not allow bending or flexing about the thickness and therefore only the flexing about the undulations 32 and 34 that define the reversed undulation 30 is allowed by which the haptic pad 38 can be moved by the sulcus, that movement being allowed by the spring flexing of the undulations 26 and 28 and nominally along the axes RA1 and RA2.

This structure of the haptics 14 provides, through operation of the reversed undulation 30 the effect of offsetting rotational force vectors that can be imposed by the sulcus pressure. Thus, the possible rotation of the lens from rotational force vectors is to be offset by the oppositely extending undulations that make up the reverse undulation.

It can be further appreciated that the reverse undulations in FIGS. 1-10 are in common order, that is they all have the first and second undulations extending laterally the same direction. This allows the installation to be visualized to avoid an upside-down installation by recognizing the order of direction of the haptics. Also, for surgical precision a visible spot 40 is printed or imprinted onto the anterior side of the haptic pads so that the surgeon can be clear of the correct orientation in implanting the IOL. That spot can be a colored dot, or a colored imprint.

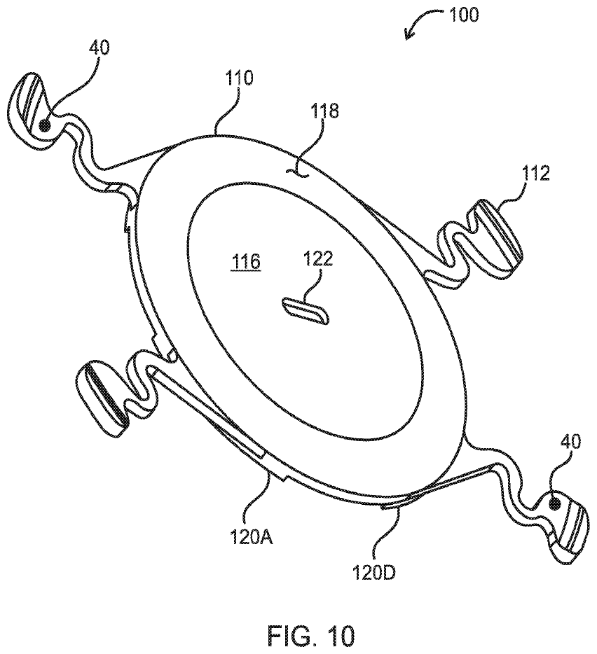

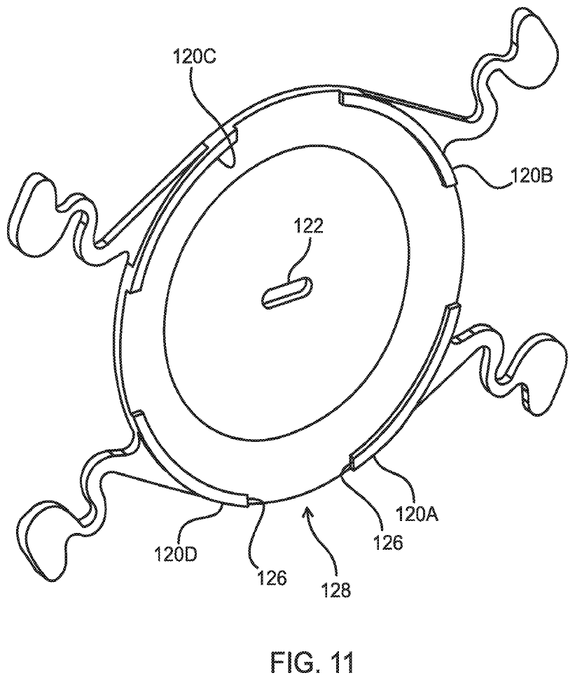

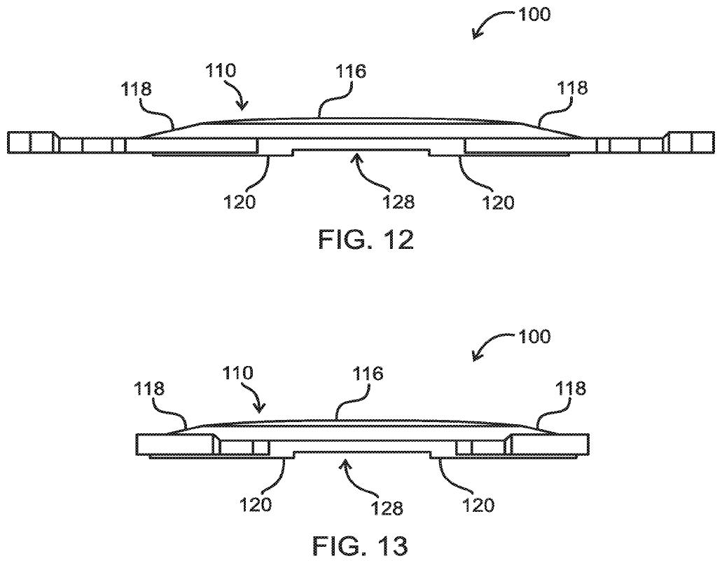

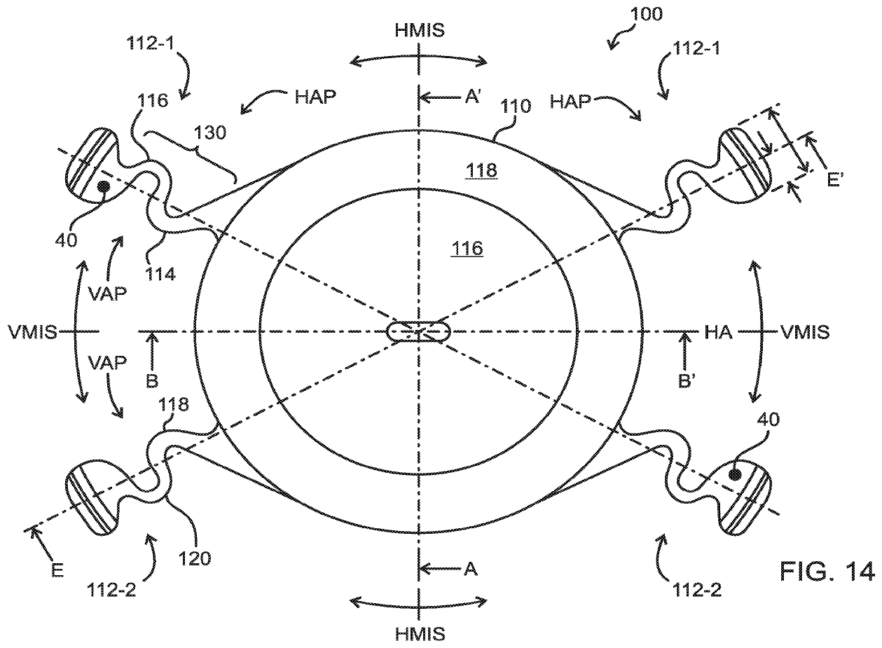

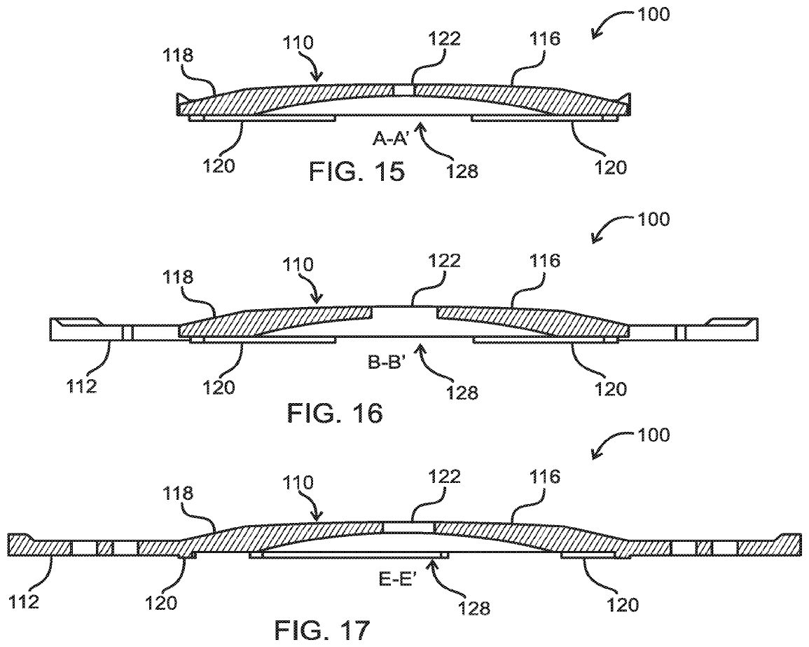

Now referring to an alternative embodiment described with relation to FIGS. 10-17. This embodiment comprises a posterior chamber phakic intraocular lens (IOL) 100 having an optic portion 110, and haptic assemblies 112 extending in pairs from the optic portion 110. In this embodiment, the optic portion 110 comprises an optic element 116 and a surround ring 118. The lens surround has an opaque surface or may be opaque through its thickness. The haptic assemblies 112 in this embodiment are configured to define associated adjacent pairs such that the reversed undulations 130 are arranged in paired opposing symmetry, that is in mirror image pairs. Thus, the vertically adjacent pair VAP having haptic 112-1 has its lower curve 114 curved toward the horizontal axis HA and its upper curve 116 away from the horizontal axis HA. The haptic 112-2 has its lower curve 118 curved toward the horizontal axis HA and its upper curve 120 curved away from the horizontal axis HA. Thus, the haptic pad in each of the opposing symmetry pairs 112-1 and 112-2 move into and out of the space allowed by the oppositely facing upper curves defining the reversed undulations 130 of each haptic. That paired relationship is defined as mirror image pairing. Thus, there is a Vertical Mirror Image Symmetry (VMIS) of haptics 112-1 and 112-2 and there is also a Horizontal Mirror Image Symmetry (HMIS) of the two haptics 112-1 above the horizontal axis HA and of the two haptics 112-2 below the horizontal axis HA. Thus, the horizontally adjacent pair HAP present the same mirror image pairing defined as a horizontally adjacent pair HAP.

With further reference to this embodiment, as best seen in FIGS. 11, 12, 13, 15, 16 and 17 on the posterior (underside) side there are rims 120 (specifically -D) which are positioned to curve around the surround ring 118 under the haptic assemblies and having interruptions 126 leaving openings 128 Then in combination with slot 123.

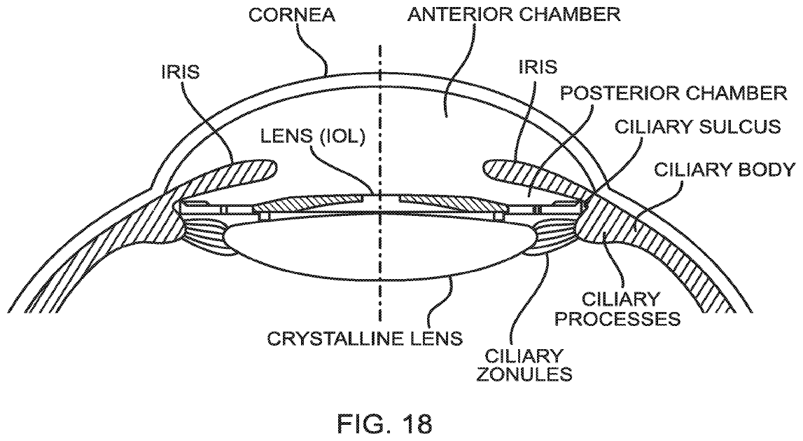

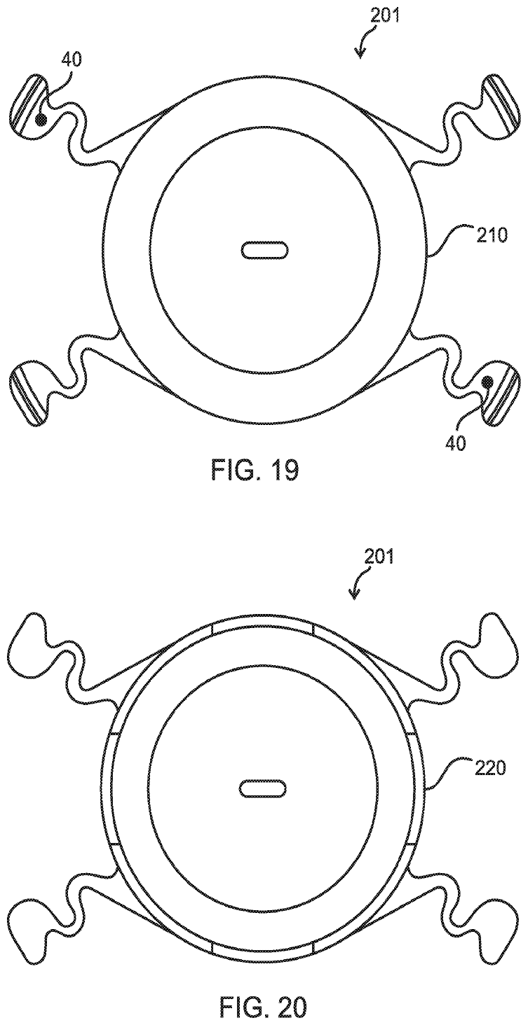

FIG. 18 is a schematic drawing showing the refractive phakic interocular lens (IOL) as in the installed position with its haptic pads in contact with the sulcus and with its rims in contact with the crystalline lens so as to be positioned above the natural crystalline lens. Thus, upon contraction of the sulcus the reversed undulations can compress and maintain the lens in correct optical alignment with the natural optical axis.

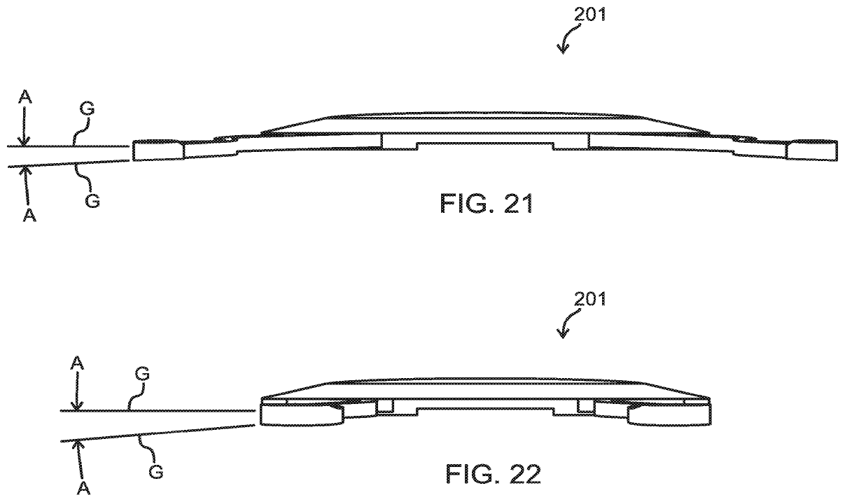

With reference to FIGS. 19-22 there is shown a further embodiment referred to as the angulated haptic embodiment 201. FIG. 19 shows the anterior surface and FIG. 20 shows the posterior surface. For illustration purposes the angulated haptic variation is shown in conjunction with the circular lens configuration as described above. However, it can be readily understood that the angulated haptic can extend from any exemplary configuration from which they extend. An anterior view is shown in FIG. 19, and a posterior view is seen in FIG. 20, however as the angle A at which the haptics extend is small, in the range of about 5 degrees to 10 degrees, the difference from a straight line extension is not apparent. Now looking however at FIGS. 21 and 22 which are respectively a top/bottom view and a side view the angulation A can be seen along lines G-G

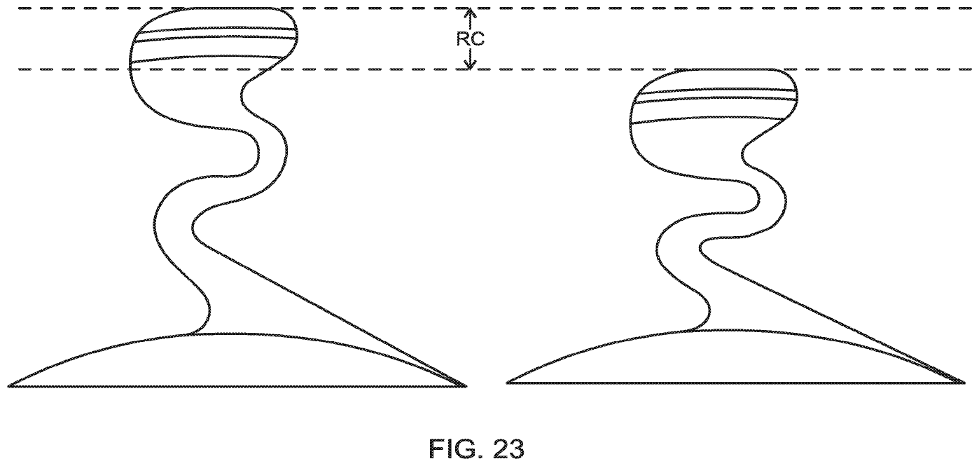

FIG. 23 illustrates the compression of the haptics showing how the reversed undulation compresses the haptic. A range of compression RC of about 3 mm would be common. This allows one size lens to fit larger or smaller sulcus sizes (diameters), sometimes refers to as sulcus to sulcus measurement.

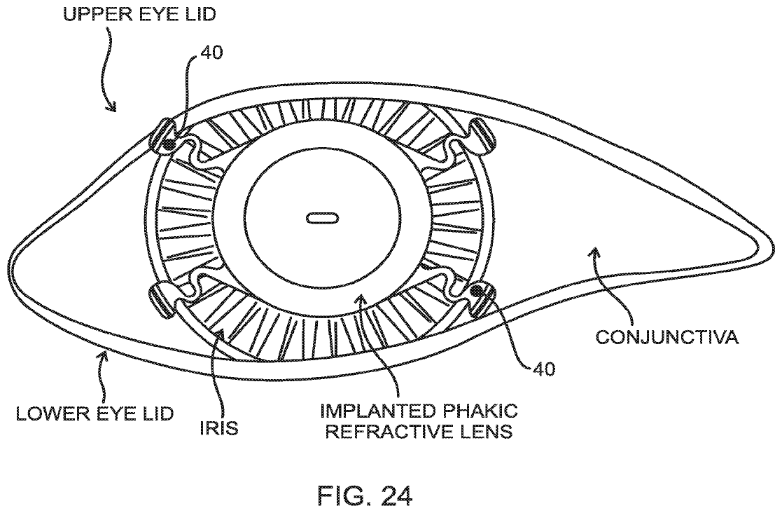

FIG. 24 shows a picture of a human eye with the IOL superimposed on it for general location purposes.

Some commonly allowed, expected or ranges of operation and construction are: Diameter across the axis E-E′ from the haptic pad sulcus surfaces 38, about 14-15 mm.

Diameter of the optic portion 10 and 110 about 7 mm.

Diameter of the optic element 16 and 116 about 5 mm.

It is considered that the foregoing described exemplary embodiments can provide a diopter range of about −1 to about −30 and +1 to +10.

The structures described above especially with relationship to the haptic configuration provide significant improvements in available use. A major aspect of that improvement is the relatively long range of the reach of the haptic pad made available by the long range of compression given by the reversed undulations. The undulations extend a small distance from the radial axis RA while allowing a relatively long radial compression range. The radial movement of the haptic is strictly controlled along the radial axis RA, allowed by the oppositely facing undulations thus provides a major advantage by allowing application to a wide range of eye anatomy, in which one haptic configuration fits a greater range of sulcus sizes than has been previously possible with a single dimensioned design that required different size lenses to be selected from. In some cases the lens was too large and bulged too much forward or other when the lens was too small was not stable in its position and moved off axis, rubbing the crystalline lens, which caused cataract.

The foregoing Detailed Description of exemplary embodiments are presented for purposes of illustration and disclosure in accordance with the requirements of the law. It is not intended to be exhaustive nor to limit the invention to the precise form(s) described, but only to enable others skilled in the art to understand how the invention may be suited for a particular use or implementation. The possibility of modifications and variations will be apparent to practitioners skilled in the art. No limitation is intended by the description of exemplary embodiments which may have included tolerances, feature dimensions, specific operating conditions, engineering specifications, or the like, and which may vary between implementations or with changes to the state of the art, and no limitation should be implied therefrom. It is intended that the scope of the invention be defined by the Claims as written and equivalents as applicable.

1. A intraocular lens (IOL) of the phakic type for phakic insertion into a recipient's eye behind the iris in the posterior chamber comprising:
a foldable optic comprising an optic member comprising an anterior optic surface, a posterior optic surface and comprising a haptic system the haptic system comprising:
a plurality of haptic assemblies attached to and extending radially away from an optical axis of the IOL the haptic assemblies comprising;
a flexible resilient member connected to the optic member and extending radially away from the optic axis in a laterally undulating form defined by radially sequential and laterally oppositely undulating portions defining a reversed undulation;
a haptic pad member extending radially from a termination of the flexible resilient member;
the plurality of haptic assemblies being oriented around the optic axis in selected angular relationship to each other.

2. The intraocular lens (IOL) of claim 1 the optic member comprising a lens having an outer diameter and concentrically to the lens extending radially from its outer diameter a darkened ring

3. The intraocular lens (IOL) of claim 1 having on a posterior surface a rim structure extending around an outer diameter of the optic member and having interruptions and having an opening at an optical axis;
whereby fluid flow is allowed through the interruptions and the opening.

4. The intraocular lens (IOL) of claim 1 the flexible resilient member of each haptic assembly comprising a reversed undulation.

5. The intraocular lens (IOL) of claim 4 wherein the reversed undulation of the flexible resilient member of each haptic assembly are in the same order of undulation.

6. The intraocular lens (IOL) of claim 4 wherein the there are four haptic assemblies defined as two vertical pairs and two horizontal pairs the vertical and horizontal pairs having shared haptic assemblies and wherein the reversed undulation of the vertical pairs are in mirror image order and the reversed undulation of the horizontal pairs are in mirror image order.

7. A haptic system for holding a posterior chamber phakic intraocular lens (IOL) having an IOL axis, the IOL having an optic member being configured to be disposed behind an iris in the eye of a patient so that the IOL axis is aligned with a visual axis of the patient the haptic system comprising:
a plurality of haptic assemblies attached to and extending radially away from an optical axis of the IOL the haptic assemblies comprising;
a flexible resilient member connected to the optic member and extending radially away from the optic axis in a laterally undulating form defined by radially sequential and laterally oppositely undulating portions defining a reversed undulation;
a haptic pad member extending radially from a termination of the flexible resilient member;
the plurality of haptic assemblies being oriented around the optic axis in selected angular relationship to each other.

8. The haptic system of claim 7 wherein the flexible resilient members of each haptic assembly are in the same undulating order.

9. The haptic system of claim 7 wherein the haptic assemblies are of an even number and are oriented at angular relationship to define horizontal and vertical pairs.

10. The haptic system of claim 7 wherein the reversed undulation of adjacent haptic assemblies are in mirror symmetry undulating order.

11. The haptic system of claim 7 having on a posterior surface of the optic member a rim structure configured to allow the IOL to contact an outer periphery of a crystalline lens and the rim structure having interruptions and having an opening at an optical axis of the optic member whereby fluid is allowed to flow through the opening and through the interruptions.

12. A method for implanting an intraocular lens, comprising:
making an incision in a cornea or sclera 2 mm or less wide; and
inserting the intraocular lens according to claim 1 into the posterior chamber through the incision;
wherein the intraocular lens will reside with the haptic pads in contact with the eye sulcus.
